(19) United States
(12) Patent Application Publication
(10) Pub. No.: US 2012/0065572 A1
(43) Pub. Date: Mar. 15, 2012

(54) Corneal crosslinking methodology using custom etched contact lenses

(76) Inventors: Scott E. Lewis, Ponte Vedra, FL (US); Jim Edwards, Lawton, OK (US); Cynthia Roberts, Columbus, OH (US); Aylin Kilic, Kavaklidere-Ankara (TR)

(21) Appl. No.: 13/136,726

(22) Filed: Aug. 9, 2011

Related U.S. Application Data
(60) Provisional application No. 61/401,201, filed on Aug. 9, 2010.

(51) Int. Cl. A61F 9/00

(52) U.S. Cl. 604/20

(57) ABSTRACT

An improved methodology for the treatment of corneal abnormalities involving the application of a crosslinking solution, such as riboflavin, to the surface of the cornea followed by exposure to UV light such that the rigidity of the cornea is increased and stabilized, the method improved by providing a transparent contact lens that is selectively etched to create areas on the lens that either preclude or reduce penetration of UV light, thereby eliminating or reducing crosslinking for areas of the cornea that do not require stabilization while allowing penetration of UV light to areas of the cornea that do require stabilization.

This application claims the benefit of U.S. Provisional Patent Application Ser. No. 61/401,201, filed Aug. 9, 2010, the disclosure of which is incorporated herein by reference.

### BACKGROUND OF THE INVENTION

This invention relates generally to treatment methodologies in the field of vision improvement for conditions where the cornea has adopted an abnormal shape. More particularly, the invention relates to methodologies incorporating corneal collagen crosslinking.

Certain vision problems are the result of changes in the shape of the cornea. Aging, disease or injury may result in the weakening of the cornea, such that the normal curvature of the cornea is replaced by localized bulging or distortion. The abnormal curvature of the cornea may change the refractive error, resulting in moderate or severe blurriness or astigmatism. Keratoconus and corneal ectasia are two common conditions that result in this abnormal curvature of the cornea. Surgical solutions to this problem include corneal transplant PKP or the insertion of small semi-circular plastic rings to increase the structural integrity of the cornea.

A relatively recent treatment methodology fort the correction of abnormal curvature problems of the cornea due to keratoconus or the like is known as corneal collagen crosslinking with riboflavin (C3-R). The C3-R method involves the application to the cornea of a riboflavin solution, or a solution of similar effect, with or without removal of the superficial epithelial barrier on the surface of the cornea, followed by exposure to UV light. Removal of the epithelial barrier is accomplished by exposure to alcohol and then physical scraping of the corneal surface. It is thought that removal of the epithelial layer optimizes the treatment, since the epithelial layer interferes to some degree with riboflavin and/or UV light penetration in the stroma. The UV light exposure in the presence of riboflavin triggers crosslinking of the collagen fibers in the cornea. The crosslinked collagen act as natural anchors to stabilize the cornea, thereby stabilizing the shape of the cornea and arresting further distortion of the cornea.

Currently, corneal problems are addressed by applying riboflavin to the entire cornea with subsequent exposure to UV light, such that the entire cornea undergoes crosslinking. In certain conditions however the problem is a localized problem whereby only limited portions of the cornea need to be stabilized by the crosslinking procedure. For example, Keratoconus (KCN) is likely driven by a disparity in corneal properties, with the “weak” areas (or areas with low modulus of elasticity) continuing to bulge as the disease progresses. It would be desirable to selectively treat only the weak regions of the cornea and not the areas that have higher elastic modulus. Therefore, in KCN it would be desirable to have greater UV penetration in the area near the bulging cone than in the surrounding areas. Ideally, the remainder of the cornea would be exposed to less UV light, and thus, although riboflavin would be present, crosslinking would either not occur (in the areas of blocked UV light) or crosslinking would occur to a lesser degree (where lesser UV light penetration is allowed). In the case of infectious keratitis, it is desirable to treat only that region of the cornea with the infection, often characterized by a corneal “melt” or area where the epithelium has eroded. Riboflavin would be applied to the entire cornea, but the UV light would be blocked to all areas except the area with an active infection, thus limiting crosslinking to this area alone. In the case of Penetrating Keratoplasty, or corneal transplant, the resulting shape is often irregular, causing visual disturbances that are not correctable with glasses or contact lenses. For these cases, it would be desirable to manipulate certain regions of the cornea, to stiffen those selected regions and ultimately create a more regular corneal shape. Areas that are targeted for stiffening would be exposed to UV light after the administration of riboflavin, and areas that do not require stiffening would be protected from UV light exposure.

It is an object of this invention to provide a methodology whereby selective crosslinking of only specific areas of the cornea can be effected while the remaining areas of the cornea are not crosslinked.

### SUMMARY OF THE INVENTION

The invention is an improved methodology for the treatment of corneal abnormalities involving the application of a crosslinking solution, such as riboflavin, to the surface of the cornea followed by exposure to UV light such that the rigidity of the cornea is increased and stabilized, the improvement comprising the step of providing a transparent contact lens that is selectively etched to create areas on the lens that either preclude or reduce penetration of UV light, thereby eliminating or reducing crosslinking for areas of the cornea that do not require stabilization while allowing penetration of UV light to areas of the cornea that do require stabilization. Preclusion of all UV light penetration is accomplished by providing a solid etched region on the contact lens. Reduction of UV light penetration is accomplished by patterned etching in the form of stripes, dots, squares or other shapes, the patterns varying in the size and spatial density of the shapes to define areas on the lens that reduce the UV penetration anywhere from 1% and 99% depending on the amount of transparent areas remaining. The contact lenses utilized in the methodology may have a standard configuration, may be provided with an enlarged vault and/or an annular rim to raise the lens off of the surface of the cornea to better entrap the crosslinking solution, or most preferably may be custom shaped using topographical techniques to better conform to the surface configuration of the cornea in order to preclude movement of the lens on the cornea during the treatment process or to biomechanically impart a desired post-crosslinking shape to the cornea.

### BRIEF DESCRIPTION OF THE DRAWINGS

FIG. 1 is a front view of a contact lens having an etched peripheral portion and a transparent central portion.

FIG. 2 is a magnified view partial view of a contact lens showing a patterned area within a larger etched of the contact lens.

### DETAILED DESCRIPTION OF THE INVENTION

In general, the invention is an improved methodology for addressing corneal abnormalities, such as for example Keratoconus (KCN), infectious keratitis, corneal ectasia or problems resulting from Penetrating Keratoplasty or injury, wherein a portion of the cornea is misshapen or weakened and it is desirable to increase the rigidity and stability of the weakened region. In a known technique, a corneal crosslinking solution, such as riboflavin, is applied to the surface of the cornea, followed by exposure to UV light. The UV light exposure results in the crosslinking of the collagen fibers in the cornea, thereby increasing the rigidity and stability of the cornea.

In a basic embodiment, the methodology of the invention comprises the steps of creating a contact lens 10 having portions of the lens surface etched to either preclude completely or reduce the passage of UV light through the lens in the etched areas 12, applying a known corneal crosslinking solution, such as riboflavin for example, to the surface of the eye, positioning the etched contact lens onto the eye, projecting UV light onto the lens such that some of the UV light penetrates the lens 10 to activate the corneal crosslinking solution, resulting in the crosslinking of selected portions of the cornea.

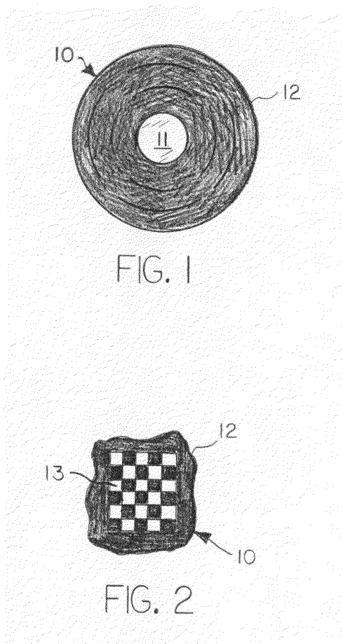

The lens 10 may be a typical contact lens of various materials, manufactured with a lathe or other lens manufacturing techniques. Portions 12 of the lens 10 are etched with a laser etching device, such as for example a CO₂ laser with a SYNRAD steering head. The resulting surface of the lens 10 has areas 12 that are etched and areas 11 that are clear of etching. The etching prohibits UV light from penetrating the contact lens. The etching is applied in various patterns determined by the amount of UV light that is to be precluded or allowed to penetrate. In areas where the UV light is to be precluded, the etched area 12 will be solid. For example, if only one relatively circular area of the cornea needs to be crosslinked, the lens 10 will be etched over its entire surface except for the circular area 11 through which the UV light is to be allowed to penetrate, as shown in FIG. 1. When the lens 10 and cornea are exposed to UV light, the corneal crosslinking solution will induce crosslinking of the collagen in the area of the cornea corresponding only to the transparent portion 11 of the lens 10, with no crosslinking occurring in the areas of the cornea corresponding to the etched portions 12 of the lens 10. In similar manner, any open configuration or multiple open configurations can be created on the lens 10 as required, such as rings, squares, curving lines, etc.

If the amount of UV light penetration is to be reduced rather than completely precluded over a given area, the lens 10 is etched with a plurality of small shapes in a predetermined and defined patterned area 13, the shapes comprising for example stripes, dots, squares or other shapes. Thus, if in a particular region of the lens 10 defined to be the patterned area 13 it is desirable to allow only 30% penetration of the UV light, then a plurality of small dots or squares, for example, are etched onto that area of the lens 10, the size and number of the dots or squares being chosen such that they cover 70% of the predetermined patterned area 13, leaving 30% of the patterned area 13 transparent, thereby reducing the amount of crosslinking in the cornea corresponding to the patterned area 13. For example, the small shapes may be presented in a checkerboard type or other regular pattern 13, as shown in FIG. 2.

In a preferred embodiment, the methodology further involves the step of taking topographical measurements or mapping the cornea surface in order to determine the necessary type or pattern of etching needed to address a particular problem. Various topography apparatuses capable of taking the measurements are well known in the field, such as those sold under the brand names SCOUT or MEDMONT for example. The equipment utilizes sophisticated software to produce data and images corresponding to the true topography of the cornea. After filtering or correcting the data set for small irregularities, the data is digitized and imported into a software application to calculate the areas of the cornea that need to be crosslinked to correct the problem.

In another preferred embodiment, the methodology further comprises the step providing a custom-shaped contact lens. The custom-shaped lens may be created so that the interior surface of the lens matches the topography of the corneal surface being treated, thereby insuring that the lens will remain stationary on the eye without rotation or slippage during the UV exposure step so that the etched portions and the transparent portions of the lens are properly situated. A custom-shaped lens may also be used to physically shape or conform an abnormally shaped cornea into a more desirable shape prior to UV exposure and crosslinking, such that after crosslinking the cornea will retain the desired shape.

This step involves taking topographical measurements or mapping the cornea surface in order to determine the three-dimensional parameters of any bulge, protrusion or other abnormal curvature of the corneal surface. Various topography apparatuses capable of taking the measurements are well known in the field, such as those sold under the brand names SCOUT or MEDMONT for example. The equipment utilizes sophisticated software to produce data and images corresponding to the true topography of the cornea. After filtering or correcting the data set for small irregularities, the data is digitized and imported into a rigid contact lens CAD/CAM software design application to calculate the lens parameters necessary to either match the present surface configuration or to reshape the cornea surface into a desired configuration. The corrected configuration is chosen for example to address vision problems resulting from corneal distortion, such that the refractive error will be reduced due to the cornea assuming a more correct configuration. For correction shaping, the lens parameters are used to create a rigid gas-permeable contact lens that will reshape the cornea surface by applying pressure against the outwardly distorted portions of the cornea, such as for example the bulge produced by keratoconus. The lens is configured to contact the cornea at the steepest region of the distortion. In the region peripheral to this point or area of maximum distortion, the lens will not contact the cornea surface, such that a partial or fully annular gap is present around the bulge. This gap allows the cornea surface to adapt into the desired curvature defined by the contact lens as the area of maximum distortion is reduced by pressure from the rigid contact lens.

The technology and methodology of topographic corneal mapping and lens shaping are discussed in more detail in our U.S. patent application Ser. No. 12/456,038, the full disclosure of which is incorporated herein by reference.

Most known corneal crosslinking procedures involve removing the epithelium using an alcohol solution or the like. However, some procedures are done via a transepithelial (TE) approach, and new compounds specifically designed for TE crosslinking are being developed. For these new compounds, it may be desirable to hold the corneal crosslinking solution in place for up to several hours prior to UV exposure in order to allow adequate penetration of the corneal crosslinking solution. For this purpose, a custom contact lens having a raised or larger vault between the lens and the cornea with a tight fitting periphery and/or an annular rim or ridge to maintain the corneal crosslinking solution on the cornea may be provided so that the patient may sit up during the pre-UV light period and be more comfortable. Openings may be provided in the lens for initial application or reapplication of the corneal crosslinking solution.

It is contemplated and understood that equivalents and substitutions for certain elements and steps set froth above may be obvious to those of ordinary skill in the art, and therefore the true scope and definition of the invention is to be as set forth in the following claims.

We claim:

1. A method of treating corneal abnormalities comprising the steps of:
providing a transparent contact lens;
etching portions of said transparent contact lens such that said contact lens comprises etched portions that preclude passage of ultraviolet light through said contact lens and transparent portions that allow passage of ultraviolet light through said contact lens;
applying a corneal crosslinking solution to a cornea, said corneal crosslinking solution inducing crosslinking of collagen within said cornea when said corneal crosslinking solution and said cornea are exposed to ultraviolet light;
positioning said contact lens on said cornea; and
exposing said corneal crosslinking solution to ultraviolet light through said contact lens for sufficient time to crosslink said collagen in the areas of said cornea corresponding to said transparent portions of said contact lens;
whereby crosslinking of said collagen is precluded in areas of said cornea corresponding to said etched portions of said contact lens.

2. The method of claim 1, wherein said step of etching portions of said contact lens comprises etching a plurality of shapes onto said lens in a patterned area, such that the amount of ultraviolet light penetrating said contact lens is reduced in said patterned area.

3. The method of claim 1, further comprising the steps of:
taking topographical measurements of the surface configuration of said cornea; and
shaping the interior surface of said contact lens to match the surface configuration of said cornea.

4. The method of claim 2, further comprising the steps of:
taking topographical measurements of the surface configuration of said cornea; and
shaping the interior surface of said contact lens to match the surface configuration of said cornea.

5. The method of claim 1, further comprising the step of:
taking topographical measurements of the surface configuration of said cornea;
determining a desired surface configuration for said cornea; and
shaping the interior surface of said contact lens such that the surface configuration of said cornea is physically reshaped into said desired configuration when said contact lens is positioned on said cornea.

6. The method of claim 2, further comprising the step of:
taking topographical measurements of the surface configuration of said cornea;
determining a desired surface configuration for said cornea; and
shaping the interior surface of said contact lens such that the surface configuration of said cornea is physically reshaped into said desired configuration when said contact lens is positioned on said cornea.

7. The method of claim 1, wherein said corneal crosslinking solution is riboflavin.

8. The method of claim 2, wherein said patterned area comprises a checkerboard pattern.

9. The method of claim 1, wherein said etching step is performed using a laser.

10. The method of claim 1, wherein said contact lens comprises a raised vault to retain said corneal crosslinking solution on said cornea.

11. The method of claim 1, wherein said contact lens comprises apertures to allow application of said corneal crosslinking solution to said cornea after the positioning of said contact lens on said cornea; and
applying additional corneal crosslinking solution to said cornea through said apertures of said contact lens after said contact lens has been positioned on said cornea.

12. A method of treating corneal abnormalities comprising the steps of:
applying a corneal crosslinking solution to a cornea, said corneal crosslinking solution inducing crosslinking of collagen within said cornea when said corneal crosslinking solution and said cornea are exposed to ultraviolet light;
positioning on said cornea a contact lens having etched portions that preclude passage of ultraviolet light through said contact lens and transparent portions that allow passage of ultraviolet light through said contact lens; and
exposing said corneal crosslinking solution to ultraviolet light through said contact lens for sufficient time to crosslink said collagen in the areas of said cornea corresponding to said transparent portions of said contact lens;
whereby crosslinking of said collagen is precluded in areas of said cornea corresponding to said etched portions of said contact lens.

13. The method of claim 12, wherein said corneal crosslinking solution is riboflavin.

14. The method of claim 12, wherein said etched portion comprises a checkerboard pattern.

15. The method of claim 12, wherein said contact lens comprises a raised vault to retain said corneal crosslinking solution on said cornea.

16. A method of treating corneal abnormalities comprising the steps of:
taking topographical measurements of the surface configuration of a cornea;
providing a transparent contact lens and shaping the interior surface of said contact lens based on the topographical measurements of the surface configuration of said cornea;
etching portions of said transparent contact lens such that said contact lens comprises etched portions that preclude passage of ultraviolet light through said contact lens and transparent portions that allow passage of ultraviolet light through said contact lens;
applying a corneal crosslinking solution to a cornea, said corneal crosslinking solution inducing crosslinking of collagen within said cornea when said corneal crosslinking solution and said cornea are exposed to ultraviolet light;
positioning said contact lens on said cornea; and
exposing said corneal crosslinking solution to ultraviolet light through said contact lens for sufficient time to crosslink said collagen in the areas of said cornea corresponding to said transparent portions of said contact lens;
whereby crosslinking of said collagen is precluded in areas of said cornea corresponding to said etched portions of said contact lens.

17. The method of claim 16, wherein said step of shaping the interior surface of said contact lens comprises matching the surface configuration of said cornea.

18. The method of claim 16, wherein said step of shaping the interior surface of said contact lens comprises determining a desired surface configuration for said cornea and shaping the interior surface of said contact lens such that the surface configuration of said cornea is physically reshaped into said desired configuration when said contact lens is positioned on said cornea.

19. The method of claim 16, wherein said step of etching portions of said contact lens comprises etching a plurality of shapes onto said lens in a patterned area, such that the amount of ultraviolet light penetrating said contact lens is reduced in said patterned area.

20. The method of claim 16, wherein said corneal crosslinking solution is riboflavin.
